(19) United States
(12) Patent Application Publication
(10) Pub. No.: US 2026/0311843 A1
(43) Pub. Date: Oct. 8, 2026

(54) ANALYSIS FRAMEWORK FOR EVALUATING HUMAN WELLNESS

(71) Applicant: West Virginia University Board of Governors on behalf of West Virginia University, Morgantown, WV (US)

(72) Inventors: Ali Rezai, Morgantown, WV (US); Victor Finomore, Morgantown, WV (US); Clay Marsh, Morgantown, WV (US)

(21) Appl. No.: 18/998,615

(22) PCT Filed: Jul. 27, 2023
(86) PCT No.: PCT/US2023/028896

§ 371 Date: Jan. 27, 2025

Related U.S. Application Data
(60) Provisional application No. 63/392,572, filed on Jul. 27, 2022.
(60) Provisional application No. 63/400,960, filed on Aug. 25, 2022.

(51) Int. Cl. G16H 20/70; G16H 10/60

(52) U.S. Cl. CPC G16H 20/70; G16H 10/60

(57) ABSTRACT

Systems and methods are provided for generating a clinical parameter for a user. A first plurality of wellness-relevant parameters representing the user are monitored at a physiological sensing device over a defined period. A second plurality of wellness-relevant parameters representing the user are obtained via a portable computing device. A third plurality of wellness-relevant parameters representing the user are retrieved from an electronic health records (EHR) system. A set of aggregate parameters are generated from the sets of wellness-relevant parameters, with each of the set of aggregate parameters comprising a unique proper subset of the parameters in the sets of wellness-relevant parameters. A clinical parameter is assigned to the user via a predictive model according to a subset of the set of aggregate parameters. An intervention is provided to the user when the clinical parameter meets a threshold value specific to the patient.

### RELATED APPLICATIONS

This is a National Stage of PCT international application PCT/US2023/028896, filed on Jul. 27, 2023, entitled “ANALYSIS FRAMEWORK FOR EVALUATING HUMAN WELLNESS”, which claims priority from each of U.S. Application No. 63/392,572, filed 27 Jul. 2022 and U.S. Application No. 63/400,960, filed 25 Aug. 2022. The subject matter of each of these applications is incorporated herein by reference in its entirety.

### TECHNICAL FIELD

This invention relates generally to assisted decision making systems and more specifically to an analysis framework for evaluating human wellness.

### BACKGROUND

Many factors affecting the health and wellness of an individual can be initially subtle and difficult to detect. Early detection of these factors can allow for effective intervention before the health and wellness of the individual is negatively impacted. In addition, it is often difficult to detect early signs of diseases or disorders that may impact the capacity of an individual for a given task or activity. In general, determinations of an individual's capacity must be performed on measurable symptoms or self-reporting, which can allow subtle decreases in capacity to go unnoticed.

### SUMMARY

In accordance with one aspect of the invention, a method is provided for generating a clinical parameter for a user. A first plurality of wellness-relevant parameters representing the user are monitored at a physiological sensing device over a defined period. A second plurality of wellness-relevant parameters representing the user are obtained via a portable computing device. A third plurality of wellness-relevant parameters representing the user are retrieved from an electronic health records (EHR) system, with the first plurality of wellness-relevant parameters, the second plurality of wellness-relevant parameters, and the third plurality of wellness-relevant parameters collectively forming a set of wellness-relevant parameters. A set of aggregate parameters are generated from the set of wellness-relevant parameters, with each of the set of aggregate parameters comprising a unique proper subset of the set of wellness-relevant parameters. A clinical parameter is assigned to the user via a predictive model according to a subset of the set of aggregate parameters. An intervention is provided to the user when the clinical parameter meets a threshold value specific to the patient.

In accordance with another aspect of the invention, a system is provided for generating a clinical parameter for a user. The system includes a physiological sensing device that monitors a first plurality of wellness-relevant parameters representing the user over a defined period and a portable computing device that obtains a second plurality of wellness-relevant parameters representing the user via a portable computing device. A network interface retrieves a third plurality of wellness-relevant parameters representing the user from an electronic health records (EHR) system, with the first plurality of wellness-relevant parameters, the second plurality of wellness-relevant parameters, and the third plurality of wellness-relevant parameters collectively forming a set of wellness-relevant parameters. A feature aggregator generates a set of aggregate parameters from set of wellness-relevant parameters, with each of the set of aggregate parameters comprising a unique proper subset of the set of wellness-relevant parameters. A predictive model assigns the clinical parameter to the user according to a subset of the set of aggregate parameters. An intervention selector provides an intervention for the user when assigned clinical parameter meets a threshold value associated with the patient. The threshold value is determined from previous clinical parameters assigned to the patient via the predictive model.

### BRIEF DESCRIPTION OF THE DRAWINGS

FIG. 1 illustrates a system for evaluating a wellness of a user in accordance with an aspect of the present invention;

FIG. 2 is a schematic example of the system of FIG. 1 using a plurality of portable monitoring devices;

FIG. 3 is a screenshot of a reaction time test from an example cognitive assessment application;

FIG. 4 is a screenshot of an attention test from an example cognitive assessment application;

FIGS. 5 and 6 are screenshots of a response inhibition test from an example cognitive assessment application;

FIG. 7 is a screenshot of a working memory (1-back) test from an example cognitive assessment application;

FIG. 8 is a screenshot of a working memory (2-back) test from an example cognitive assessment application;

FIG. 9 illustrates a method for evaluating a wellness of a user; and

FIG. 10 is a schematic block diagram illustrating an exemplary system of hardware components.

### DETAILED DESCRIPTION

The term “wellness” as used herein in intended to refer to the mental, physical, cognitive, behavioral, social, and emotional health of a user and should be construed to cover each of the health, function, balance, resilience, homeostasis, disease, and condition of the user. In various examples herein, the wellness of the user can be related to the readiness of the user to perform job-related, athletic, or everyday functions, enter into a flow or zone state, the susceptibility of the user to an infectious disease, the ability of the user to resist the effects of addiction, worsening pain of a user, increased or reduces stress, anxiety, a quality of life of the user, and similar qualities of a user. The wellness of the user is referred informally herein as a status of a “Human Operating System.”.

A “wellness-relevant parameter” is a parameter that is relevant to the wellness of a user.

A “biological rhythm” is any chronobiological phenomenon that affects human beings, including but not limited to, circadian rhythms, ultradian rhythms, infradian rhythms, diurnal cycle, sleep/wake cycles, and patterns of life.

A “portable monitoring device,” as used herein, refers to a device that is worn by, carried by, or implanted within a user that incorporates either or both of an input device and user interface for receiving input from the user and sensors for monitoring either a wellness-relevant parameter or a parameter that can be used to calculate or estimate a wellness-relevant parameter.

An “index”, as used herein, is intended to cover composite statistics derived from a series of observations and used as an indicator or measure. An index can be an ordinal, continuous, or categorical value representing the observations and correlations, and should be read to encompass statistics traditionally referred to as “scores” as well as the more technical meaning of index.

A “clinical parameter”, as used herein, can be any continuous or categorical parameter representing the mental, neurological, physical, cognitive, behavioral, social, and emotional health of a user and can represent any or all of the health, function, a zone or flow state, balance, resilience, homeostasis, disease, and condition of the user.

A “neurological” parameter represents one or more of motor function, sensory function, the status of cranial nerves, the spinal cord, and the brain stem, and cortical and sub-cortical functions.

An “physiological sensing device,” as used herein, is a device to measure one or more physiological parameters and/or biological rhythms. A physiological sensing device is often implanted, ingested, or wearable, although in some instances an off-body device can be used to capture physiological parameters.

A “portable computing device,” as used herein, is a computing device that can carried by the user, such as a smartphone, smart watch, tablet, notebook, and laptop, that can measure a wellness-relevant parameter either through sensors on the device or via interaction with the user. A portable computing device can include, for example, a user interface for receiving an input from the user, kinematic sensors for measuring activity by the user, and location services that track a location of the user.

As used herein, a “predictive model” is a mathematical model or machine learning model that either predicts a future state of a parameter or estimates a current state of a parameter that cannot be directly measured.

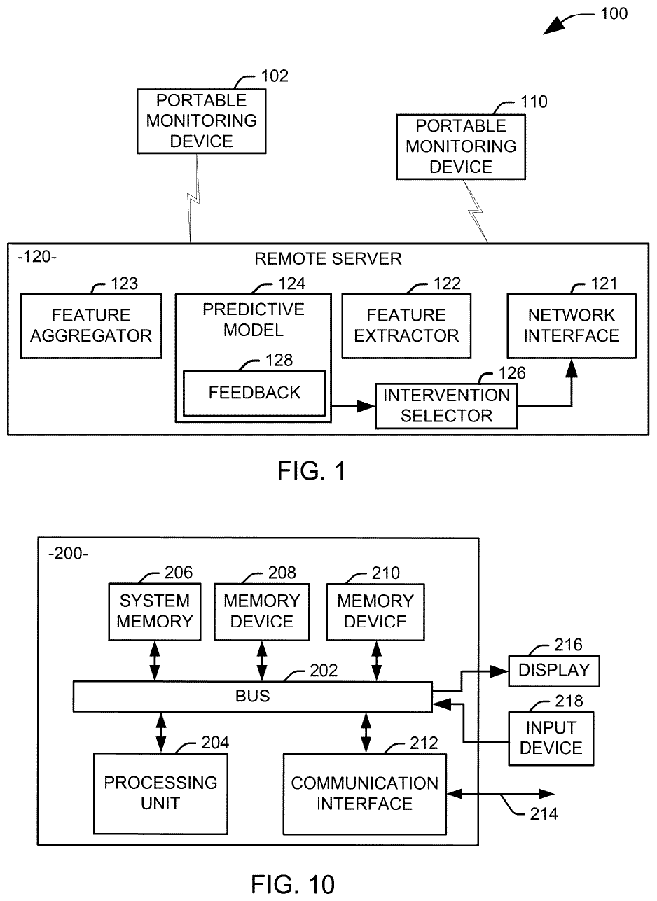

FIG. 1 illustrates a system 100 for evaluating a wellness of a user in accordance with an aspect of the present invention. The user can be a patient under care for a disorder, a caregiver of a patient under care for a disorder (e.g., a family member caring for a person with Alzheimer's) who supplies data about the patient to generate wellness parameters and interventions, as well as a caregiver or other individual who is monitored for stress, burnout, and declines in function. A user can also be an individual who has not yet been diagnosed with a given disorder, but who is considered to be at risk for the disorder. One example of an application to which the system 100 can be applied can be found in a co-pending U.S. patent application, filed on May 1, 2023 with Ser. No. 18/310,465 and entitled “Screening, Monitoring, and Treatment Framework for Focused Ultrasound”. The entire contents of this application are hereby incorporated by reference.

The system 100 includes a plurality of portable monitoring devices 102 and 110 that include sensors for monitoring systems tracking wellness-relevant parameters for the user. It will be appreciated that a given portable monitoring device (e.g., 102) can either communicate directly with a remote server 120 to provide the wellness-relevant parameters to the server or with another portable monitoring device (e.g., 110) that relays the wellness-relevant parameters to the server. In one example, the plurality of portable monitoring devices can include a physiological sensing device and a portable computing device. By using portable monitoring devices 102 and 110, measurements can be made continuous from any of a user's home, classroom, job, or sports field—literally anywhere from the battlefield to the board room. Additional parameters can be either retrieved from an electronic health records (EHR) interface and/or other available databases via a network interface 121 associated with the server 120. These parameters can include, for example, employment information (e.g., title, department, shift), age, sex, home zip code, genomic data, nutritional information, medication intake, household information (e.g., type of home, number and age of residents), social and psychosocial data, consumer spending and profiles, financial data, food safety information, the presence or absence of physical abuse, and relevant medical history.

As noted above, wellness-relevant parameters can include at least physiological, cognitive, motor/musculoskeletal, sensory, sleep, biomarkers and behavioral parameters. A physiological parameter is a parameter relating to the chemical, electrical, or mechanical function of the body. Table I provides non-limiting examples of physiological parameters that can be measured and exemplary tests, devices, and methods, to measure the physiological parameters.

TABLE I

| Physiological | Exemplary Devices and Methods to |
|---|---|
| Parameter | Measure Physiological Parameters |
| Brain Activity | Electroencephalogram, Magnetic Resonance |
| | Imaging, including functional Magnetic |
| | Resonance Imaging (fMRI), PET, SPECT, |
| | MEG, near-infrared spectroscopy, functional |
| | near-infrared spectroscopy, and other |
| | brain imaging modalities looking at |
| | electrical, blood flow, neurotransmitter, |
| | and metabolic function |
| Heart rate | Electrocardiogram and Photoplethysmogram |
| Heart rate variability | Electrocardiogram, Photoplethysmogram |
| Ocular Imaging & Eye | Optical Coherence Tomography Angiography, |
| tracking | Optos retinal imaging, Pupillometry, |
| | including tracking saccades, fixations, |
| | and pupil size (e.g., dilation) |
| Perspiration | Perspiration sensor |
| Blood pressure | Sphygmomanometer |
| Body temperature | Thermometer, infrared thermography |
| Blood oxygen saturation | Pulse oximeter/accelerometer |
| and respiratory rate | |
| Skin conductivity | Electrodermal activity |
| Facial emotions | Camera or EMG based sensors for emotion |
| | and wellness |
| Sympathetic and | Derived from the above measurements |
| parasympathetic tone | |

The physiological parameters can be measured via wearable or implantable devices as well as self-reporting by the user via applications in a mobile device, which facilitates measuring these physiological parameters in a naturalistic, non-clinical setting. For example, a smart watch, ring, or patch can be used to measure the user's heart rate, heart rate variability, body temperature, blood oxygen saturation, movement, and sleep. These values can also be subject to a diurnal analysis to estimate variability and reviewed in view of expected changes due to biological rhythms, as well as deviations from an expected pattern of biological rhythms. For example, the biological rhythms of a user can be tracked for a predetermined period (e.g., ten days), to establish a normal pattern of biological rhythms. Oscillations in biological rhythms can be detected as departures from this established pattern.

It will be appreciated that each of the physiological parameters can be measured regularly (e.g., at five-minute intervals) to form a time series for each parameter. This time series can be used as generated, normalized according to a measure of central tendency and measure of deviation for a predetermined time window (e.g., a week) for the user, or converted to a frequency domain or set of wavelet coefficients. In one example, the raw signal used to determine a parameter, such as heart rate variability, can be divided into different frequency bands and separate time series can be generated for each frequency band. Using the example of heart rate variability measured using a photoplethysmography (PPG) sensor, the output of PPG sensor can be divided into a high frequency band centered at about 0.08 Hz and a low frequency band centered at around 0.00008 Hz, and separate time series can be derived from the data in each band.

Cognitive parameters are parameters relating to the mental function of the patient, including, for example, memory, decision making, and attention. Table II provides non-limiting examples of cognitive parameters that are gamified and that can be measured and exemplary methods and tests/tasks to measure such cognitive parameters. The cognitive parameters can be assessed by a battery of cognitive tests that measure, for example, executive function, decision making, working memory, attention, and fatigue.

TABLE II

| | Exemplary Tests and Methods to |
|---|---|
| Cognitive Parameter | Measure Cognitive Parameters |
| Temporal discounting | Kirby Delay Discounting Task |
| Alertness and fatigue | Psychomotor Vigilance Task |
| Focused attention and | Erikson Flanker Task |
| response inhibition | |
| Working memory | N-Back Task |
| Attentional bias towards | Dot-Probe Task |
| emotional cues | |
| Inflexible persistence | Wisconsin Card Sorting Task |
| Decision making | Iowa Gambling Task |
| Risk taking behavior | Balloon Analogue Risk Task |
| Inhibitory control | Anti-Saccade Task |
| Sustained attention | Sustained Attention |
| Executive function | Task Shifting or Set Shifting Task |

These cognitive tests can be administered in a clinical/laboratory setting or in a naturalistic, non-clinical setting such as when the user is at home, work or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch.

TABLE III provides non-limiting examples of parameters associated with movement and activity of the user, referred to herein alternatively for ease of reference as “motor parameters,” that can be measured and exemplary tests, devices, and methods. The use of portable monitoring, physiological sensing, and portable computing devices allows the motor parameters to be measured. Using embedded accelerometer, GPS, and cameras, the user's movements can be captured and quantified to see how wellness affects them and related to the wellness-relevant parameters.

TABLE III

| Motor/Musculoskeletal | Exemplary Tests and Methods to Measure |
|---|---|
| Parameter | Motor/Musculoskeletal Parameters |
| Activity level | Daily movement total, time of activities, |
| | from wearable accelerometer, steps, Motion |
| | Capture data, gait analysis, GPS, deviation |
| | from established geolocation patterns, |
| | force plates |
| Gait analysis | Gait mat, camera, force plats |
| Range of motion | Motion capture, camera, |

TABLE IV provides non-limiting examples of parameters associated with sensory acuity of the user, referred to herein alternatively for ease of reference as “sensory parameters,” that can be measured and exemplary tests, devices, and methods.

TABLE IV

| | Exemplary Tests and Methods to |
|---|---|
| Sensory Parameter | Measure sensory Parameters |
| Vision | Visual acuity test, visual field tests, |
| | eye tracking, EMG |
| Hearing | Hearing tests |
| Touch | Two-point discrimination, frey filament |
| Smell/taste | “Scratch-and-sniff” test, “sip, spit, |
| | and rinse test” |
| Vestibular | Vestibula function test |
| Introception | Heartbeat detection test, questionnaires |

TABLE V provides non-limiting examples of parameters associated with a sleep quantity and quality of the user, referred to herein alternatively for ease of reference as “sleep parameters,” that can be measured and exemplary tests, devices, and methods.

TABLE V

| | Exemplary Tests and Methods |
|---|---|
| Sleep Parameter | to Measure Sleep Parameters |
| Sleep from wearables | Sleep onset & offset, sleep quality, sleep |
| | quantity, from wearable accelerometer, |
| | temperature, and PPG, |
| Sleep Questions | Pittsburg Sleep Quality Index, Functional |
| | Outcomes of Sleep Questionnaire, Fatigue |
| | Severity Scale, Epworth Sleepiness Scale |
| Devices | Polysomnography; ultrasound, camera, bed |
| | sensors |
| Circadian Rhythm | Light sensors, actigraphy, serum levels, |
| | core body temperature |

TABLE VI provides non-limiting examples of parameters extracted by locating biomarkers associated with the user, referred to herein alternatively for ease of reference as “biomarker parameters,” that can be measured and exemplary tests, devices, and methods. Biomarkers can also include imaging and physiological biomarkers related to a state of chronic wellness and improvement or worsening of the chronic wellness state.

TABLE VI

| | Exemplary Tests and Methods to |
|---|---|
| Biomarkers Parameter | Measure Biomarkers Parameters |
| Genetic biomarkers | Genetic testing |
| Immune biomarkers | Blood, saliva, and/or urine tests |
| including TNF-alpha, | |
| immune alteration (e.g., | |
| ILs), oxidative stress, and | |
| hormones (e.g., cortisol) | |
| Gut microbiome | Stool sample analysis |

Table VII provides non-limiting examples of psychosocial and behavioral parameters, referred to herein alternatively for ease of reference as “psychosocial parameters,” that can be measured and exemplary tests, devices, and methods.

TABLE VII

| Psychosocial or | Exemplary Tests and Methods to Measure |
|---|---|
| Behavioral Parameter | Psychosocial or Behavioral Parameters |
| Symptom log | Presence of specific symptoms (i.e., |
| | fever, headache, cough, loss of smell) |
| Medical Records | Medical history, prescriptions, setting |
| | for treatment devices such as spinal |
| | cord stimulator, imaging data |
| Wellness Rating | Visual Analog Scale, Defense & Veterans |
| | wellness rating scale, wellness scale, |
| | Wellness Assessment screening tool |
| | and outcomes registry |
| Burnout | Burnout inventory or similar |
| Physical, Mental, | User-Reported Outcomes Measurement |
| and Social Health | Information System (PROMIS), Quality |
| | of Life Questionnaire |
| Depression | Hamilton Depression Rating Scale |
| Anxiety | Hamilton Anxiety Rating Scale |
| Mania | Snaith-Hamilton Pleasure Scale |
| Mood/ | Profile of Mood States; Positive |
| Catastrophizing scale | Affect Negative Affect Schedule |
| Affect | Positive Affect Negative |
| | Affect Schedule |
| Impulsivity | Barratt Impulsiveness Scale |
| Adverse Childhood | Childhood trauma |
| Experiences | |
| Daily Activities | Exposure, risk taking |
| Daily Workload | NASA Task Load Index, Perceived |
| and Stress | Stress Scale (PSS), |
| | Social Readjustment Rating |
| | Scale (SRRS) |
| Social Determents | Social determents of health |
| of Health | questionnaire |

The behavioral and psychosocial parameters can measure the user's functionality as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. A smart device, such as a smartphone, tablet, or personal computer can be used to administer the subjective/self-reporting questionnaires. Using embedded accelerometers and cameras, these smart devices can also be used to capture the facial expression analysis to analyze the user's facial expressions that could indicate mood, anxiety, depression, agitation, and fatigue.

The remote server 120 analyzes the data collected by the portable monitoring devices 102 and 110 and any clinical data received from the EHR system at the network interface 121. The remote server 120 can be implemented as a dedicated physical server or as part of a cloud server arrangement. In addition to the remote server, data can be analyzed, in whole or in part, on the local device itself and/or in a federated learning mechanism, in which case, any data from the EHR can be provided to the local device via an appropriate network interface. Information received from the portable monitoring devices 102 and 110 and the network interface 121, is provided to a feature extractor 122 that extracts a plurality of features. These features are aggregated at a feature aggregator 123 to provide a set of aggregate parameters. The aggregate parameters are then either used directly as features for a predictive model 124 or used to derive features for the predictive model.

The feature extractor 122 determines categorical and continuous parameters representing the wellness-relevant parameters. In one example, the parameters can include descriptive statistics, such as measures of central tendency (e.g., median, mode, arithmetic mean, or geometric mean) and measures of deviation (e.g., range, interquartile range, variance, standard deviation, etc.) of time series of the monitored parameters, as well as the time series themselves. It will be appreciated that the descriptive statistics can be taken across multiple time frames to represent acute changes (i.e., changes over hours to days), sub-acute changes (i.e., changes over days to weeks), or chronic changes (i.e., changes over months and years). Specifically, the feature set provided to the predictive model can include, for at least one parameter, either two values representing the value for the parameter at different times or a single value, such as a measure of central tendency or a measure of deviation which represents values for the parameter across a plurality of times. In addition, the model can combine multiple users to interact together to refine the prediction, such as a social model of spouse, children, family, co-workers, friends and others.

In other examples, the features can represent departures of the user from an established pattern for the features. For example, values of a given parameter can be tracked overtime, and measures of central tendency can be established, either overall or for particular time periods. The collected features can represent a departure of a given parameter from the measure of central tendency, such as a Z-score or another normalized or non-normalized measure of deviation. For example, changes in the activity level of the user, measured by either or both of kinematic sensors and global positioning system (GPS) tracking can be used as a wellness-relevant parameter. Additional elements of monitoring can include the monitoring of the user's compliance with the use of a smart phone, TV, portable device, a portable device. For example, a user may be sent messages by the system inquiring on their wellness level, general mood, or the status of any other wellness-relevant parameter on the portable computing device. A measure of compliance can be determined according to the percentage of these messages to which the user responds via the user interface on the portable computing device.

In one implementation, the feature extractor 122 can perform a wavelet transform on a time series of values for one or more parameters to provide a set of wavelet coefficients. It will be appreciated that the wavelet transform used herein is two-dimensional, such that the coefficients can be envisioned as a two-dimensional array across time and either frequency or scale.

For a given time series of parameters, xᵢ, the wavelet coefficients, Wₐ(n), produced in a wavelet decomposition can be defined as:

$${W}_{a} ( n ) = {a}^{- 1} {\sum }_{i = 1}^{M} {x}_{i} ψ ( \frac{i - n}{a} ) \qquad Eq . 3$$

wherein ψ is the wavelet function, M is the length of the time series, and a and n define the coefficient computation locations.

The feature extractor 122 can also include a facial expression classifier (not shown) that evaluates recorded data from a camera and/or recorded images or videos of the user's face from one of the portable monitoring devices 102 and 110, such as a smartphone or other mobile device, to assign an emotional state to the user at various times throughout the day. The extracted features can be categorical, representing the most likely emotional state of the user, or continuous, for example, as a time series of probability values for various emotional states (e.g., anxiety, discomfort, anger, etc.) as determined by the facial expression classifier. The feature extractor 122 can also include one or more image classifiers that reduce provided medical images to categorical or continuous features for use at the predictive model. It will be appreciated that each of the facial expression classifier and the one or more image classifiers can be implemented using one or more of the models discussed below for use in the predictive model 124.

The feature aggregator 124 generates a set of aggregate parameters from the set of wellness-relevant parameters collected by the portable monitoring devices 102 and 110 and any clinical data received from the EHR system at the network interface 121. It will be appreciated that each aggregate parameter can be a weighted combination of the set of wellness-relevant parameters or functions of parameters from the set of wellness-relevant parameters. Accordingly, a given aggregate parameter can represent a plurality of wellness-relevant parameters, and, in general, the plurality of wellness-relevant parameters represented by each aggregate parameter will be related, such that the aggregate parameter represents a specific domain of wellness for the user. In general, each aggregate parameters can use parameters from various sources, such that a given aggregate parameter can be a combination of features from two or more of the portable monitoring devices 102 and 110 and the network interface 121. In some implementations, the system 100 can include multiple predictive models (not shown) that each receive a unique proper subset of the aggregate parameters. Each predictive model can provide a different clinical parameter representing a different aspect of the user's wellness, such that the aggregate parameters can be utilized for multiple purposes in evaluating the wellness of the user.

The predictive model 124 can utilize one or more pattern recognition algorithms, each of which analyze the extracted features or a subset of the extracted features to assign a continuous or categorical clinical parameter to the user. In one example, the predictive model 124 can assign a continuous parameter that corresponds to a likelihood that that user has or is about to contract a specific disease or disorder, a likelihood that the user is experiencing the effects of aging, a likelihood that the user is experiencing an onset of dementia, a likelihood that the user has or will develop a neurodegenerative disorder, a likelihood that the user will experience an intensifying of symptoms, or “flare-up,” of a chronic condition, a likelihood that the user will use an addictive substance during rehabilitation or treatment, a current or predicted level of pain for the user, an expected performance level of the user associated with a current or future time for a particular activity or occupation, a change in symptoms associated with a disease or disorder, a current or predicted response to treatment, a likelihood that the user has experienced an increase in stress, or an overall wellness level of the user. In another example, the predictive model 124 can assign a categorical parameter that corresponds to ranges of the likelihoods described above, the presence or predicted presence of a specific disease or disorder, a set of categories representing the users readiness for a particular activity or occupation, categories representing changes in symptoms associated with a disease or disorder (e.g., “improving”, “stable, “worsening”), categories representing a current or predicted response to treatment, categories representing a status of the user (e.g., normal,” “stressed”, “μl”), or categories indicating that a particular action should be suggested to the user. The generated parameter can be stored in a non-transitory computer readable medium, for example, as part of a record in an electronic health records database, or used to suggest a course of action to the user.

Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. The training process can be accomplished on a remote system and/or on the local device or wearable, app. The training process can be achieved in a federated or non-federated fashion. For rule-based models, such as decision trees, domain knowledge, for example, as provided by one or more human experts or extracted from existing research data, can be used in place of or to supplement training data in selecting rules for classifying a user using the extracted features. Any of a variety of techniques can be utilized for the classification algorithm, including support vector machines, regression models, self-organized maps, fuzzy logic systems, data fusion processes, boosting and bagging methods, rule-based systems, or artificial neural networks.

Federated learning (aka collaborative learning) is a predictive technique that trains an algorithm across multiple decentralized edge devices or servers holding local data samples, without exchanging their data samples. This approach stands in contrast to traditional centralized predictive techniques where all data samples are uploaded to one server, as well as to more classical decentralized approaches which assume that local data samples are identically distributed. Federated learning enables multiple actors to build a common, robust predictive model without sharing data, thus addressing critical issues such as data privacy, data security, data access rights, and access to heterogeneous data. Its applications are spread over a number of industries including defense, telecommunications, IoT, or pharmaceutics.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier. Another example is utilizing an autoencoder to detect outlier in wellness-relevant parameters as an anomaly detector to identify when various parameters are outside their normal range for an individual.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used.

In one implementation, the predictive model 124 can include a constituent model that predicts future values for the aggregate parameters, such as a convolutional neural network that is provided with one or more two-dimensional arrays of wavelet transform coefficients as an input. The wavelet coefficients detect changes not only in time, but also in temporal patterns, and can thus reflect changes in the ordinary biological rhythms of the user. It will be appreciated that a given constituent model can use data in addition to the aggregate parameters, such as other extracted features and user data to provide these predictions. Additionally, or alternatively, the predictive model 124 can use constituent models that predict current or future values for the aggregate parameters, with these measures then used as features for generating the output of the predictive model. This data can also be used to group the user with users who respond similarly to these parameters, with data fed back from users within a given group used to better tailor the model to the user.

The model can also be used to facilitate an intervention strategy to one or more designated recipients, which can include the user, a health care provider, family, friends, social system, a care team, a supervisor, a coach, a caretaker, and other entities, in which a course of action is suggested, for example, in response to a change in the wellness of the individual. To this end, an intervention selector 126 can provide an intervention for any user for which the assigned clinical parameter indicates that invention would be desirable, for example, any undesired deviation of the clinical parameter from a desired range of values. For example, the intervention selector 126 can communicate with the user or an individual assisting with the care of the user via the network interface 121. Examples of disorders for which detection or the disorder or a heightened risk of the disorder can trigger intervention include, but are not limited to, anxiety, depression, suicidal thoughts, stress, mood, agitation, obsessions, compulsion, OCD, Parkinson's, tremor, chronic pain, arrythmias, heart failure, syncope, diabetes, hypertension, vascular disorders, pulmonary disorders, inflammatory disorders, obesity, stroke, autoimmune disorder, endocrine disorders, neurodegenerative disorders, neuropsychiatric disorders, inflammation, cardiovascular, diabetes, mood and developmental disorders, musculoskeletal disorders, decline in human performance, (acute, subacute, and chronic changes) and myocardial infarction. The intervention can include information provided from the system for raising awareness of a detected issue or education on that issue, initiation or modification of a current treatment including but not limited to medications, neuromodulation of the brain surface, the deep brain, the spinal cord, or peripheral nerves via electrical or magnetic stimulation, ultrasound, or light, ultrasound lesioning, blood brain barrier opening in combination with therapeutics, radiation treatment, heart catheterization, electrophysiology procedures, biologicals, surgical intervention, behavioral and social intervention, digital intervention via portable device, a care provider coming to the individual, directing an individual to go to a clinic, emergency room, or hospital, or directing the user to obtain additional testing. For example, when a user is predicted to experience cardiac distress during activity, the user can be instructed to cease the activity and seek medical attention. The selected intervention can also be a change in the parameters used for an existing intervention, (e.g., a change in the dosage of a medication, parameters used for neuromodulation, a frequency of neuromodulation or another therapy, or a frequency of therapy or coaching). In one implementation, each of a plurality of interventions can be represented by individual thresholds for a continuous clinical parameter, such that less resource-intensive or invasive interventions can be selected for patients who meet the first threshold, and more resource-intensive or invasive interventions can be provided for patients who meet the second threshold.

It will be appreciated that a suggested course of action can be any course of action intended to enhance the wellness of the user or others and can include, for example, taking a prescribed meditation, performance of prescribed exercises, cessation of a current activity, contacting a medical professional, and quarantining. In one example, when the user is determined to be experiencing significant stress, the intervention can include a “digital reset” in which the user is instructed to engage in deep breathing or other stress reduction techniques. If this is ineffective or if the problem reoccurs, the intervention can be elevated to a digital intervention prescribed by a medical expert, in which the user engages in guided stress reduction techniques as periodic intervals. If this is also ineffective, the user can be instructed to seek the assistance of a medical professional, caregiver, counselor, coach, peer advisor, or therapist, or that individual can be instructed to contact the user. It will be appreciated that the model is predictive, and thus interventions for stress, pain, and similar issues can be suggested before the user is even aware of the issue. In another example, a user can be instructed in sleep hygiene in response to indications of inadequate or restless sleep, directed to engage in a sleep study, or referred to a physician for analysis. It will be appreciated that interventions associated with sleep can also be assigned for other detected issues, such as decreased cognitive function, decreased athletic, job, or other performance, or heightened risks of stroke and heart disease.

It will be appreciated that the system 100 can provide multiple clinical parameters or thresholds for a single clinical parameter to detect changes over multiple time scales. For example, a first clinical parameter or patient-specific threshold can detect acute changes, which are changes over the course of hours or days. A second parameter or patient-specific threshold can detect subacute changes, that is, changes over the course of days or weeks. A third clinical parameter or patient-specific threshold can detect chronic changes, that is, changes over the course of months or years. This allows for both acute events and gradual decline in the wellness of the user to be detected and appropriate interventions to be assigned. It will be appreciated that the relative value of the detection of acute, sub-acute, and chronic changes will vary with the disorder being monitored.

In some implementations, the predictive model 124 can include a feedback component 128 can tune various parameters of the predictive model 124 based upon the accuracy of predictions made by the model. In one example, the feedback component 128 can be shared by a plurality of predictive models 124, with the outcomes for users associated with each predictive model compared to the outcomes predicted by the output of the model. Parameters associated with the model, such as thresholds for producing categorical inputs or outputs from continuous values, can be adjusted according to the differences in the actual and predicted outcomes. In one example, a continuous output of the system can be compared to a threshold value to determine if the user is increase or decrease in wellness related parameters. This threshold can be varied by the feedback model 128 to increase the accuracy of the determination.

In practice, the thresholds used for a given model can be determined initially from data collected from other patients. As additional patient data is gathered over time to establish a set of baseline data, the thresholds can be refined to be specific to the user, representing a meaningful change in the value of the clinical parameter for that patient. Where multiple thresholds are used, representing, for example, different interventions or time scales, each of these thresholds can be refined according to the collected patient data. For interventions, outcome data can also be collected and used to refine the thresholds for a patient, with the thresholds for interventions determined to be ineffective for a given patient adjusted to suggest them less frequently, and the thresholds for interventions determined to be effective changed to suggest these interventions more frequently.

Alternatively, the predictive model 124 can obtain feedback at the level of the individual model. For example, in a predictive model 124 using constituent models to predict future values of wellness-relevant parameters, the model receives consistent feedback as to the accuracy of these predictions once the wellness-relevant parameter is measured. This feedback can be used to adjust parameters of the model, including individualized thresholds for that user to produce categorical inputs or outputs from continuous values, or baseline values for biological rhythms associated with the user. Alternatively, feedback can be provided from a final output of the model and compared to other data, such as a user-reported status (e.g., wellness level), to provide feedback to the model. In one implementation, a reinforcement learning approach can be used to adjust the model parameters based on the accuracy of either predicted future values of wellness-relevant parameters at intermediate stages of the predictive model 124 or the output of the predictive model. For example, a decision threshold used to generate a categorical output from a continuous index produced by the predictive model 124 can be set at an initial value based on feedback from a plurality of models from previous users and adjusted via the reinforcement model to generate a decision threshold specific to the user.

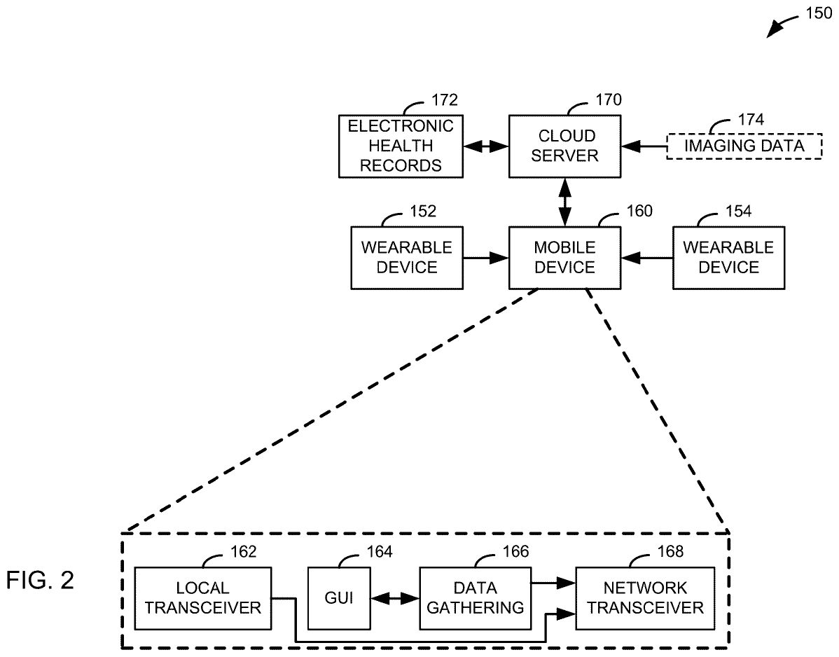

FIG. 2 is a schematic diagram 150 of one example of the system of FIG. 1 using a plurality of portable monitoring devices 152, 154, and 160. In the illustrated implementation, the first and second portable monitoring devices 152 and 154 are wearable devices, worn on the wrist and finger, respectively. Wellness-relevant parameters monitored by the first and second portable monitoring devices 152 and 154 can include, for example, heart rate, heart rate variability, metrics of sleep quality, biological, and circadian rhythm variations, metrics of sleep quantity, physical activity of the user, body orientation, movement, arterial blood pressure, respiratory rate, peripheral arterial oxyhemoglobin saturation, as measured by pulse oximetry, maximum oxygen consumption, temperature, and temperature variation. Wearable devices, as used herein, can include any wearable items implemented with appropriate sensors, including watches, wristbands, rings, headbands, headbands, and other wearable items that can maintain sensors in an appropriate position for monitoring the wellness-relevant parameters. It will be appreciated that a given wearable device 152 and 154 can monitor many of these parameters with great frequency (e.g., every five minutes) allowing for a detailed time series of data to be generated.

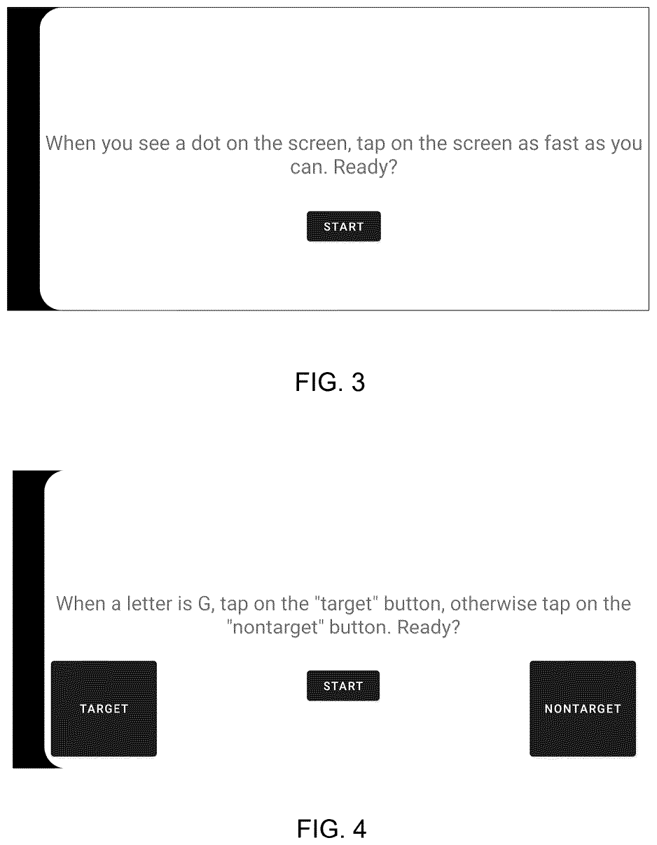

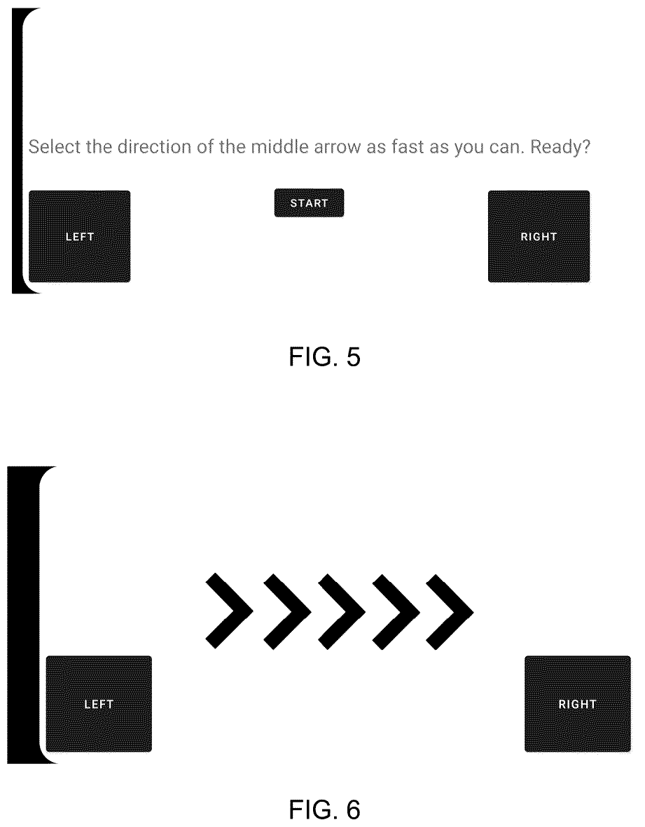

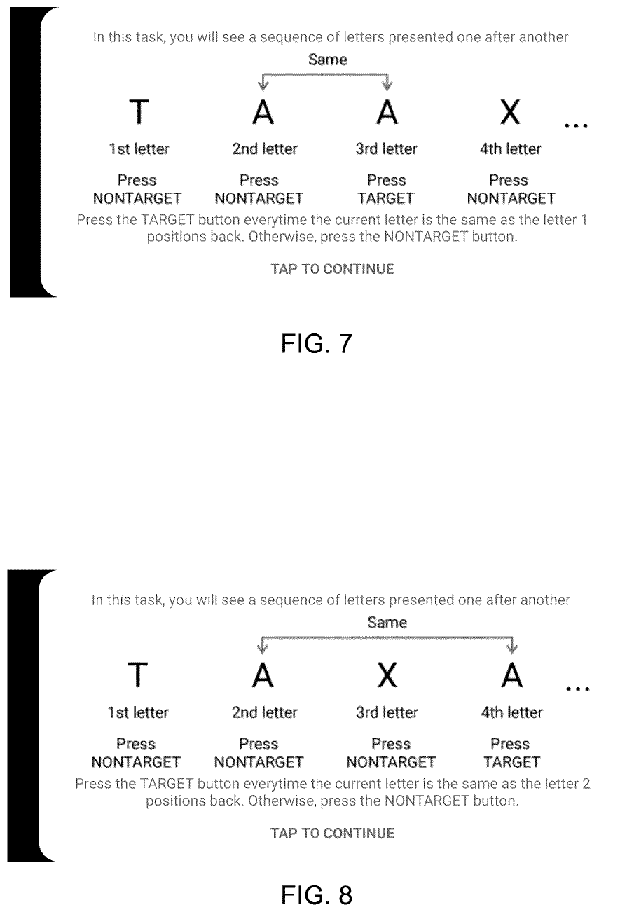

The system 150 can further include a mobile device 160 that communicates with the first and second portable monitoring devices 152 and 154 via a local transceiver 162. The mobile device 160 can also include a graphical user interface 164 that allows a user to interact with one or more data gathering applications 166 stored at the base unit. One example of a possible data gathering applications can include a cognitive assessment application that tests various measures of cognitive function. These can include working memory, attention, and response inhibition, fatigue, cognition. Further, these metrics can be compared to an established baseline to estimate a measure of fatigue for the user. Screenshots from an example cognitive assessment application are provided as FIGS. 3-8. Another data gathering application can include a questionnaire application that allows the user to self-report wellness, mood, mental, physical, and emotional states, and stress. In general, the data gathering applications 166 can be selected and configured to monitor each of:
1. Attention, alertness, and fatigue
2. Memory
3. Mental flexibility
4. Mood & Emotion
5. Perceptual processing
6. Sensory acuity
7. Motor function
8. Neuro capacity
9. Social network
10. Social systems
11. Wellness rating
12. Wellness location
13. Wellness
14. Alertness
15. Medical and treatment history
16. Return to work, improvement of cognitive, motor, sensory and behavioral function quality of life and function.

The mobile device 160 further comprises a network transceiver 168 via which the system 150 communicates with a cloud server 170 via an Internet connection. It will be appreciated that the cloud server 170 can be designed and configured to be HIPAA compliant, allowing for storage of patient data at the server 170. In this example, the cloud server 170 includes a feature aggregator that generates a set of seven aggregate parameters from a set of wellness-relevant parameters provided from the first and second portable monitoring devices 152 and 154, the mobile device 160, and data stored in one or more databases, including an electronic health records (EHR) system 172. In one example, a standardized set of queries can be sent to the EHR system 172 to retrieve data for generating some or all of the seven aggregate parameters. The feature aggregator can also generate wellness-relevant parameters from imaging data 174 representing the patient, for example, using a convolutional neural network or similar deep learning model to extract one or more numerical or categorical parameters representing the imaging data.

In the illustrated implementation, the seven aggregate parameters include a first aggregate parameter representing autonomic function of the user, a second aggregate parameter representing a cognitive function of the user, a third aggregate parameter representing a motor and musculoskeletal health of the user, a fourth aggregate parameter representing sleep disruptions and general disruptions of circadian rhythm, a fifth aggregate parameter representing relevant biomarkers and generic information identified for the user, a sixth aggregate parameter representing sensory function and changes in function for the user, and a seventh parameter representing a sociobehavioral health of the user. These aggregate parameters, or values derived from these aggregate parameters, can be employed at the predictive model to produce a clinical parameter associated with the patent. It will be appreciated, however, that in other implementations, these parameters can be reduced, added to, or substituted with other aggregate parameters, such as parameters representing neurological function, endocrine function, gastrointestinal function, genitourinary function, cardiac function, vascular health, pulmonary function, and inflammatory status.

In the illustrated example, the cloud server 170 hosts a predictive model, for example, implemented as a recurrent neural network, specifically a network with a long short-term memory architecture. In this example, the aggregate parameters can be provided to the predictive model as time series along with other relevant data. An output of the model is an index representing the current level of overall wellness being experienced by the user. It will be appreciated that the index can be used for clinical studies to determine a response to treatment or responses to stimuli. Further, the index can be used to recommend a course of action for the user, such as contacting a medical professional, taking prescribed medications, engaging in meditation or another calming activity, or quarantining.

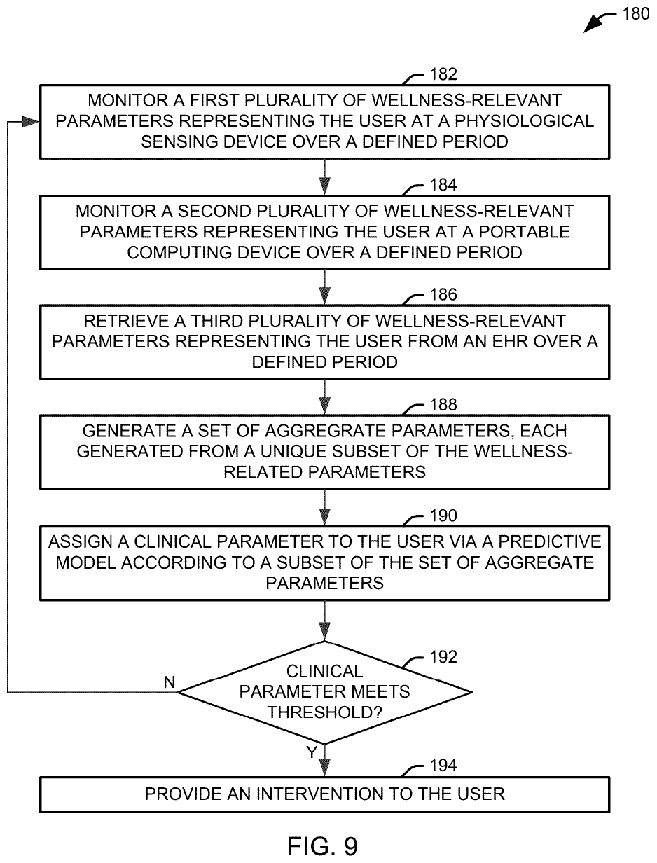

FIG. 9 illustrates a method 180 for evaluating wellness for a user. The result of the method is a clinical parameter representing some aspect of a wellness of the user. At 182, a first plurality of wellness-relevant parameters representing the user are monitored at a physiological sensing device over a defined period. In one example, the first plurality of wellness-relevant parameters can include parameters representing the autonomic function of the user and parameters representing the sleep and circadian rhythms of the user. At 184, a second plurality of wellness-relevant parameters representing the user are obtained via a portable computing device. In one example, the second plurality of wellness-relevant parameters can include parameters representing the cognitive and/or sociobehavioral wellness of the user. At 186, a third plurality of wellness-relevant parameters representing the user are retrieved from an electronic health records (EHR) system. The third plurality of wellness-relevant parameters can include, for example, parameters representing the musculoskeletal health, genomics, and various biomarkers of the user. The first plurality of wellness-relevant parameters, the second plurality of wellness-relevant parameters, and the third plurality of wellness-relevant parameters collectively form a set of wellness-relevant parameters.

At 188, a set of aggregate parameters are generated from the set of wellness-relevant parameters, with each of the set of aggregate parameters comprising a unique proper subset of the set of wellness-relevant parameters. In one example, the set of aggregate parameters includes at least a first aggregate parameter representing autonomic function of the user, a second aggregate parameter representing a cognitive function of the user, and a third aggregate parameter representing a motor and musculoskeletal health of the user. In another example, the set of aggregate parameters includes at least a first aggregate parameter representing sleep and circadian rhythms of the user, a second aggregate parameter representing a sociobehavioral function of the user, and a third aggregate parameter representing a biomarkers and genomics of the user.

At 190, a clinical parameter is assigned to the user via a predictive model according to a subset of the set of aggregate parameters. In one example, the clinical parameter is a value representing an overall wellness of the user, and the subset of the set of aggregate parameters comprises the entire set of aggregate parameters. In another example, the subset of the set of aggregate parameters is a proper subset. It will be appreciated that the aggregate parameters can be provided to multiple predictive models, with each predictive model receiving a unique subset of the set of aggregate parameters. In one example, the predictive model is an anomaly detection model, which detects deviations from expected values within a feature space and determines when these deviations are significant. The anomaly detection model can be trained on data from the user, which establishes a baseline of expected values for the user, or on data collected from other users. In one example, training the predictive model is initially trained on data collected from other users while values for the subset of the set of wellness-relevant parameters are collected from the user over a period of time. Once a sufficient amount of data is available for the user, the predictive model is retrained on the collected values for the subset of the set of wellness-relevant parameters.

In one example, a wavelet decomposition is performed on the time series for at least one aggregate parameter to provide a set of wavelet coefficients, and the set of wavelet coefficients or one or more values derived from the set of wavelet coefficients can be provided to the predictive model. Additionally or alternatively, the user can be assigned a predicted value representing a future value of a given aggregate parameter according to the values for the subset of aggregate parameters, and the value assigned to the user can be assigned based on the predicted value.

Additionally or alternatively, feedback, in the form of a self-reported wellness level from the user or a measured future value for a parameter, can be used to refine the predictive model. For example, the self-reported or measured value can be compared to the value assigned to the user via a predictive model, and a parameter associated with the predictive model can be changed according to the comparison. In one example, this can be accomplished by generating a reward for a reinforcement learning process based on a similarity of the measured outcome to the value assigned to the user and changing the parameter via the reinforcement learning process.

At 192, it is determined if the clinical parameter meets a threshold value. If not (N), the method returns to 182 to continue monitoring the user. If the clinical parameter meets the threshold value (Y), the method advances to 194, where an intervention is provided to the user. For example, any of the wellness-related parameters, aggregate parameters, and the value assigned to the user can be provided, for example, via a user interface or network interface, to one or more of the user, the user's health care provider, the user's care team, a research team, a user's workplace, a user's sports team, an insurer, or other interested entities. This allows the value to be used to make decisions about the user's care and activities and can also be used to request that a provider or other individual involved in the user's care contact the user. Messages provided to the user can be used to improve the user's awareness, perception and interpretation of being in an overall positive and negative states, allowing the user to learn strategies for avoiding negative states and inducing positive states. The provided wellness data can also be used for improvement or optimization of cognitive, motor, sensory, and behavioral function as well as generally attempting to improve the user's quality of life through suggesting actions for the user in response to changes in the clinical parameter. For example, a message can be transmitted to the user's portable computing device suggesting a course of action for the user when the clinical parameter meets a specific threshold value.

In one implementation, the steps at 182, 184, 186, 188, and 190 can be repeated to establish a set of baseline data for the user. Once the baseline data is established, at least one threshold value associated with the clinical parameter can be determined from the set of baseline user data that is specific to the user, representing values of the clinical parameter that would be abnormal for that specific user. Once the threshold or thresholds are established, a novel set of wellness-relevant parameters can be obtained, and a novel set of aggregate parameters can be generated from the novel set of wellness-relevant parameters. A novel clinical parameter, representing a current state of the user, can be generated at the predictive model from a subset of the novel set of aggregate parameters and compared to the one or more thresholds. The intervention is provided to the user if the novel clinical parameter meets the determined threshold. As the monitoring continues, the threshold can be further refined according to collected parameter data and user outcomes.

FIG. 10 is a schematic block diagram illustrating an exemplary system 200 of hardware components capable of implementing examples of the systems and methods disclosed herein. The system 200 can include various systems and subsystems. The system 200 can be a personal computer, a laptop computer, a workstation, a computer system, an appliance, an application-specific integrated circuit (ASIC), a server, a server BladeCenter, a server farm, etc.

The system 200 can include a system bus 202, a processing unit 204, a system memory 206, memory devices 208 and 210, a communication interface 212 (e.g., a network interface), a communication link 214, a display 216 (e.g., a video screen), and an input device 218 (e.g., a keyboard, touch screen, and/or a mouse). The system bus 202 can be in communication with the processing unit 204 and the system memory 206. The additional memory devices 208 and 210, such as a hard disk drive, server, standalone database, or other non-volatile memory, can also be in communication with the system bus 202. The system bus 202 interconnects the processing unit 204, the memory devices 206-210, the communication interface 212, the display 216, and the input device 218. In some examples, the system bus 202 also interconnects an additional port (not shown), such as a universal serial bus (USB) port.

The processing unit 204 can be a computing device and can include an application-specific integrated circuit (ASIC). The processing unit 204 executes a set of instructions to implement the operations of examples disclosed herein. The processing unit can include a processing core.

The additional memory devices 206, 208, and 210 can store data, programs, instructions, database queries in text or compiled form, and any other information that may be needed to operate a computer. The memories 206, 208 and 210 can be implemented as computer-readable media (integrated or removable), such as a memory card, disk drive, compact disk (CD), or server accessible over a network. In certain examples, the memories 206, 208 and 210 can comprise text, images, video, and/or audio, portions of which can be available in formats comprehensible to human beings.

Additionally or alternatively, the system 200 can access an external data source or query source through the communication interface 212, which can communicate with the system bus 202 and the communication link 214.

In operation, the system 200 can be used to implement one or more parts of a system for evaluating the wellness of a user in accordance with the present invention. Computer executable logic for implementing the system resides on one or more of the system memory 206, and the memory devices 208 and 210 in accordance with certain examples. The processing unit 204 executes one or more computer executable instructions originating from the system memory 206 and the memory devices 208 and 210. The term “computer readable medium” as used herein refers to a medium that participates in providing instructions to the processing unit 204 for execution. This medium may be distributed across multiple discrete assemblies all operatively connected to a common processor or set of related processors. Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to, portable or fixed storage devices, optical storage devices, wireless channels, and/or various other storage mediums capable of storing that contain or carry instruction(s) and/or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A method for generating a clinical parameter for a user, the method comprising:
performing the following until a set of baseline user data is generated:
monitoring a first plurality of wellness-relevant parameters representing the user at a physiological sensing device over a defined period;
obtaining a second plurality of wellness-relevant parameters representing the user via a portable computing device;
retrieving a third plurality of wellness-relevant parameters representing the user from an electronic health records (EHR) system, the first plurality of wellness-relevant parameters, the second plurality of wellness-relevant parameters, and the third plurality of wellness-relevant parameters collectively forming a set of wellness-relevant parameters;
generating a set of aggregate parameters from the set of wellness-relevant parameters, each of the set of aggregate parameters comprising a unique proper subset of the set of wellness-relevant parameters; and
assigning a clinical parameter to the user via a predictive model according to a subset of the set of aggregate parameters;
determining a threshold value associated with the clinical parameter from the set of baseline user data;
obtaining a novel set of wellness-relevant parameters;
generating a novel set of aggregate parameters from the novel set of wellness-relevant parameters;
generating a novel clinical parameter via the predictive model, representing a current state of the user, from a subset of the novel set of aggregate parameters; and
providing an intervention to the user if the novel clinical parameter meets the determined threshold.

2. The method of claim 1, wherein providing an intervention to the user comprises providing one of the novel set of wellness-relevant parameters, the novel set of aggregate parameters, and the novel clinical parameter to one of a medical professional, a caregiver, a therapist, a peer advisor, and a coach.

3. The method of claim 1, wherein the clinical parameter is a value representing an overall wellness of the user, and the subset of the set of aggregate parameters comprises the entire set of aggregate parameters.

4. The method of claim 1, wherein the providing the intervention to the user comprises reporting the clinical parameter to one of a health care provider, an insurance company, a care team, a research team, a coach of the user, and a workplace of the user via a network interface.

5. The method of claim 1, wherein the providing the intervention to the user comprises transmitting a message to the user suggesting a that guides the user through a stress reduction technique.

6. The method of claim 1, wherein the providing the intervention to the user comprises providing the intervention to the user via portable computer device.

7. The method of claim 1, wherein the user is a first user of a plurality of users, and the predictive model is an anomaly detection model trained on data collected from the plurality of users.

8. The method of claim 1, wherein the user is a first user of a plurality of users, the method further comprising:
training the predictive model on data collected from the plurality of users; and
collecting values for the set of wellness-relevant parameters from the first user over a period of time; and
retraining the predictive model on the collected values for the set of wellness-relevant parameters.

9. The method of claim 8, wherein retraining the predictive model on the subset of wellness-relevant parameters comprises retraining the predictive model via a reinforcement learning process.

10. The method of claim 1, wherein the set of aggregate parameters comprises a first aggregate parameter representing autonomic function of the user, a second aggregate parameter representing a cognitive function of the user, and a third aggregate parameter representing a motor and musculoskeletal health of the user.

11. The method of claim 1, wherein generating the set of aggregate parameters from the set of wellness-relevant parameters comprises generating a time series for one of the set of aggregate parameters and assigning the clinical parameter to the user via the predictive model comprises performing a wavelet decomposition on the time series of the one of the set of aggregate parameters to provide a set of wavelet coefficients, and assigning the value according to at least the set of wavelet coefficients and the subset of the set of aggregate parameters.

12. The method of claim 1, wherein assigning the clinical parameter to the user via the predictive model comprises:
assigning the user a predicted value representing a future value of one of the subset of the set of aggregate parameters according to the set of wellness-relevant parameters and at least one previously determined value for the one of the set of aggregate parameters; and
assigning the clinical parameter to the user according to at least the predicted value for the one of the subset of the set of aggregate parameters.

13. The method of claim 1, wherein the set of aggregate parameters includes at least a first aggregate parameter representing sleep and circadian rhythms of the user, a second aggregate parameter representing a sociobehavioral function of the user, and a third aggregate parameter representing a biomarkers and genomics of the user.

14. A system for generating a clinical parameter for a user, the system comprising:
a physiological sensing device that monitors a first plurality of wellness-relevant parameters representing the user over a defined period;
a portable computing device that obtains a second plurality of wellness-relevant parameters representing the user via a portable computing device;
a network interface that retrieves a third plurality of wellness-relevant parameters representing the user from an electronic health records (EHR) system, the first plurality of wellness-relevant parameters, the second plurality of wellness-relevant parameters, and the third plurality of wellness-relevant parameters collectively forming a set of wellness-relevant parameters;
a feature aggregator that generates a set of aggregate parameters from the set of wellness-relevant parameters, each of the set of aggregate parameters comprising a unique proper subset of the set of wellness-relevant parameters;
a predictive model that assigns the clinical parameter to the user according to a subset of the set of aggregate parameters; and
an intervention selector that provides an intervention for the user when assigned clinical parameter meets a threshold value associated with the patient, the threshold value being determined from previous clinical parameters assigned to the patient via the predictive model.

15. The system of claim 14, wherein the predictive model is a recurrent neural network.

16. The system of claim 14, wherein at least one of the second plurality of wellness-relevant parameters is derived from psychosocial assessment data provided by the user, the portable computing device comprising a user interface that allows the user to interact with a psychosocial assessment application.

17. The system of claim 14, further comprising a feedback component that collects values for the set of wellness-relevant parameters from the user over a period of time and adjusts the threshold value associated with the patient according to the collected values for the set of wellness-relevant parameters.

18. The system of claim 14, wherein the set of aggregate parameters comprises a first aggregate parameter representing autonomic function of the user, a second aggregate parameter representing a cognitive function of the user, a third aggregate parameter representing a motor and musculoskeletal health of the user, a fourth aggregate parameter representing sleep disruptions and general disruptions of circadian rhythm, a fifth aggregate parameter representing relevant biomarkers and generic information identified for the user, a sixth aggregate parameter representing sensory function and changes in function for the user, and a seventh parameter representing a sociobehavioral health of the user.

19. The system of claim 14, further comprising a feature extractor that performs a wavelet decomposition on a time series of values for the one of the set of wellness-related parameters to provide a set of wavelet coefficients, the feature aggregator generating the set of aggregate parameters from according to at least the set of wavelet coefficients and the set of wellness-related parameters.

20. The system of claim 14, further comprising a feedback component that collects values for the set of wellness-relevant parameters from the user over a period of time, collecting values representing an outcome for the user, and retraining the predictive model on the collected values for the set of wellness-relevant parameters and the values representing the outcome for the user.
